(19) United States
(12) Patent Application Publication
(10) Pub. No.: US 2005/0192376 A1
(43) Pub. Date: Sep. 1, 2005

(54) Denture care method and kit

(76) Inventors: Thinh Nguyen Ha, Cincinnati, OH (US); Barbra Ann Cappel, Monroe, OH (US); Jayanth Rajaiah, Loveland, OH (US); Tao He, Mason, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 10/787,804

(22) Filed: Feb. 26, 2004

(51) Int. Cl. A61K 6/00

(52) U.S. Cl. 523/120

(57) ABSTRACT

A denture care kit may comprise a denture adhesive composition, a container, and information for use. The denture adhesive composition may comprise a safe and effective adhesive amount of a denture adhesive component, wherein the denture adhesive composition may have a viscosity of from about 5 to about 80 Brookfield. The container may be used for holding and dispensing the denture adhesive composition. The container may comprise a nozzle. The nozzle may have a tip diameter of from about 80 mils to about 120 mils, and a length from about 10 mm to about 25 mm. Information for use for decreasing food entrapment may be used to instruct the denture wearer to apply the denture adhesive composition as a continuous peripheral bead along at least one of the back quarter portion, the back half portion, the back three-quarters portion, the front quarter portion, the front half portion, the front three-quarters portion, or the entire periphery of an upper and/or lower denture.

### TECHNICAL FIELD

This invention relates to the field of applying denture adhesive composition to a denture.

### BACKGROUND OF THE INVENTION

Ordinary removable dentures comprise teeth mounted in a suitable plate or base. Denture stabilizers may be used to fill the interstices between the dentures and the gums and/or oral mucous tissues. Prior to placement of the denture on the gums and/or oral mucous tissues, a denture stabilizer may be applied to the denture surface, which, for a perfect fit, should uniformly contact the gums and/or oral mucous tissues. The denture stabilizer may be formulated not only for its adherent properties, but also to provide a cushion or gasket between the denture and the gums and/or oral mucous tissues, thereby positioning the denture securely on the gums and/or oral mucous tissues.

Considerable effort has been made over the years to develop improved denture adhesive compositions. Both synthetic and natural polymers and gums have been used alone, in combination, and in combination with various other adhesives and other materials in an attempt to improve hold and reduce oozing of the adhesive from under the denture, and to reduce messiness and difficulty of removing the residual adhesive from the mouth and dentures. For example, alkyl vinyl ether-maleic copolymers and salts thereof are known for providing adequate hold in denture adhesive compositions. Such disclosures include: U.S. Pat. No. 3,003,988, Germann et al., issued Oct. 10, 1961; U.S. Pat. No. 4,980,391, Kumar et al., issued Dec. 25, 1990; U.S. Pat. No. 5,073,604, Holeva et al., issued Dec. 17, 1991; U.S. Pat. No. 5,525,652, Clarke, issued Jun. 11, 1996; U.S. Pat. No. 5,340,918, Kittrell et al., issued Aug. 23, 1994; U.S. Pat. No. 5,830,933, Synodis et al., issued Nov. 3, 1998.

In addition to adhesion, it may be desirable to minimize the often painful entrapment of food (e.g., nuts, seeds, kernels, etc.) between the denture wearer's gums and/or oral mucous tissues and the denture. The point of entry between the denture wearer's gum and/or oral mucous tissues and the denture normally occurs around the perimeter of the denture, especially at the back portion of the denture. Thus, a need exists for a denture adhesive composition which provides not only improved hold, but also reduces the entrapment of food between the gums and/or oral mucous tissues and the denture.

### SUMMARY OF THE INVENTION

A denture care kit may comprise a denture adhesive composition, a container, and information for use. The denture adhesive composition may comprise a safe and effective adhesive amount of a denture adhesive component. The container may be used for holding and dispensing the denture adhesive composition. The container may comprise a nozzle. Information for use for decreasing food entrapment may be used to instruct the denture wearer to apply the denture adhesive composition as a continuous peripheral bead to an upper and/or lower denture.

A method may comprise opening a box having information fixed and/or printed within an interior portion of the box. The information may instruct the denture wearer how to apply a denture adhesive composition along the periphery of a denture in a manner that will decrease food entrapment between the denture and the denture wearer's gums and/or oral mucous, tissues. The method may further comprise removing a tube from the box. The tube may contain the denture adhesive composition. The method may further comprise applying a continuous peripheral bead of the denture adhesive composition to an upper and/or a lower denture in substantial conformity with the information.

### BRIEF DESCRIPTION OF THE DRAWINGS

FIG. 1 is a top view of an upper denture having a continuous peripheral bead of denture adhesive composition around its entire top surface such that an unbroken ring is formed.

FIG. 2 is a bottom view of a lower denture having a continuous peripheral bead of denture adhesive composition around its entire bottom surface such that an unbroken ring is formed.

FIG. 3 is a top view of an upper denture having a continuous peripheral bead of denture adhesive composition around the back quarter portion of its top surface.

FIG. 4 is a bottom view of a lower denture having a continuous peripheral bead of denture adhesive composition around the back quarter portion of its bottom surface.

FIG. 5 is a top view of an upper denture having a continuous peripheral bead of denture adhesive composition around the back half portion of its top surface.

FIG. 6 is a bottom view of a lower denture having a continuous peripheral bead of denture adhesive composition around the back half portion of its bottom surface.

FIG. 7 is a top view of an upper denture having a continuous peripheral bead of denture adhesive composition around the periphery of the back three-quarters portion of its top surface.

FIG. 8 is a bottom view of a lower denture having a continuous peripheral bead of denture adhesive composition around the back three-quarters portion of its bottom surface.

FIG. 9 is a top view of an upper denture having a continuous peripheral bead of denture adhesive composition around the front quarter portion of its top surface.

FIG. 10 is a bottom view of a lower denture having a continuous peripheral bead of denture adhesive composition around the front quarter portion of its bottom surface.

FIG. 11 is a top view of an upper denture having a continuous peripheral bead of denture adhesive composition around the front half portion of its top surface.

FIG. 12 is a bottom view of a lower denture having a continuous peripheral bead of denture adhesive composition around the front half portion of its bottom surface.

FIG. 13 is a top view of an upper denture having a continuous peripheral bead of denture adhesive composition around the front three-quarters portion of its top surface.

FIG. 14 is a bottom view of a lower denture having a continuous peripheral bead of denture adhesive composition around the front three-quarters portion of its bottom surface.

FIG. 15 is a top view of an upper denture having a continuous peripheral bead of denture adhesive composition around its top surface such that an unbroken ring is formed. Non-peripheral beads of denture adhesive composition are also oriented in the central portion of the denture.

FIG. 16 is a bottom view of a lower denture having a continuous peripheral bead of denture adhesive composition around its bottom surface such that an unbroken ring is formed. A non-peripheral bead of denture adhesive composition is also oriented in the central portion of the denture.

FIG. 17 is an exploded perspective view of a container, nozzle, and lid.

FIG. 18 is an exploded view of a box holding a container of denture adhesive composition.

FIG. 19 is a top view of an unfolded box for holding a denture adhesive composition.

FIG. 20-A is a perspective view of a partially opened box for holding a denture adhesive composition.

FIG. 20-B is a perspective view of an opened box for holding a denture adhesive composition.

### DETAILED DESCRIPTION OF THE INVENTION

The term “AVE/MA” as used herein refers to alkyl vinyl ether-maleic acid or anhydride copolymer. The term “AVE/MA/IB” refers to terpolymers with alkyl vinyl ether, maleic acid or anhydride, and isobutylene. The term “mixed polymer salts” or “mixed salts”, as used herein, refers to salts of AVE/MA and/or salts of AVE/MA/IB where at least 2 different cations are mixed on the same polymer with each other or with other salts.

The phrase “back half portion” with regard to the upper denture means about the back half portion of the top surface, illustrated by area L3 in FIG. 5. The phrase “back half portion” with regard to the lower denture means about the back half portion of the bottom surface, illustrated by area L4 in FIG. 6.

The phrase “back quarter portion” with regard to the upper denture means about the back quarter portion of the top surface, illustrated by area L1 in FIG. 3. The phrase “back quarter portion” with regard to the lower denture means about the back quarter portion of the bottom surface, illustrated by area L2 in FIG. 4.

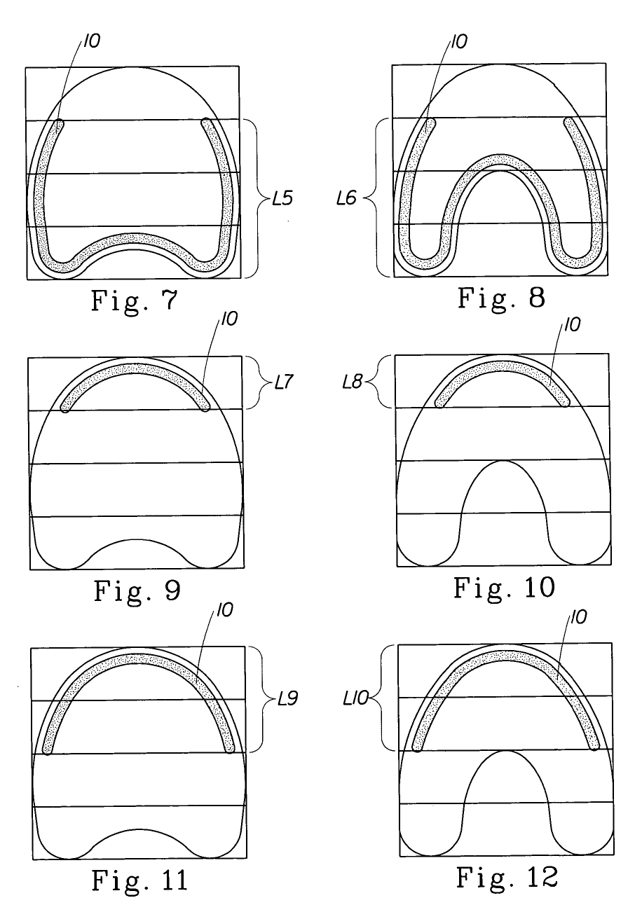

The phrase “back three-quarters portion” with regard to the upper denture means about the back three-quarters portion of the top surface, illustrated by area L5 in FIG. 7. The phrase “back three-quarters portion” with regard to the lower denture means about the back three-quarters portion of the bottom surface, illustrated by area L6 in FIG. 8.

The term “free acid” or “FA” component, as used herein, refers to the unreacted carboxyl groups (—COOH) of AVE/MA copolymer and/or AVE/MA/IB. The term “free acid” or “FA” component also refers to any monovalent cations of carboxyl groups (e.g., COONa) of AVE/MA copolymer and/or AVE/MA/IB. Monovalent cations may include Group IA cations, such as sodium, potassium, hydrogen, etc.

The phrase “front half portion” with regard to the upper denture means about the front half portion of the top surface, illustrated by area L9 in FIG. 11. The phrase “front half portion” with regard to the lower denture means about the front half portion of the bottom surface, illustrated by area L10 in FIG. 12.

The phrase “front quarter portion” with regard to the upper denture means about the front quarter portion of the top surface, illustrated by area L7 in FIG. 9. The phrase “front quarter portion” with regard to the lower denture means about the front quarter portion of the bottom surface, illustrated by area L8 in FIG. 10.

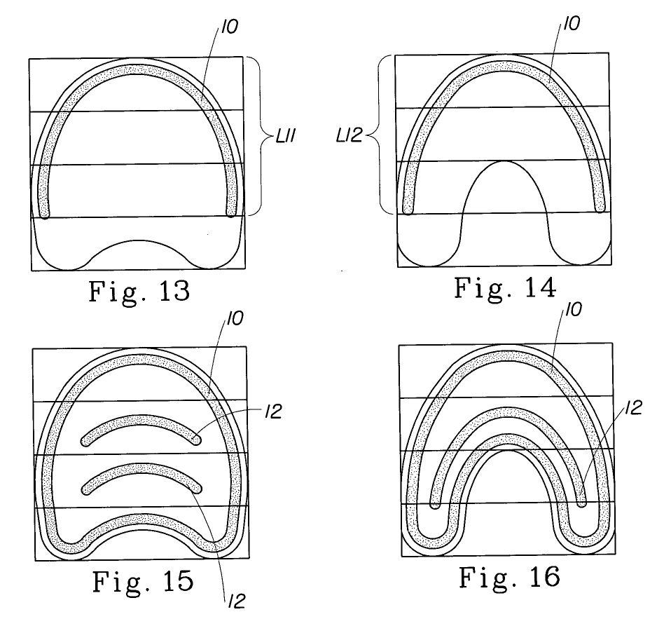

The phrase “front three-quarters portion” with regard to the upper denture means about the front three-quarters portion of the top surface, illustrated by area L11 in FIG. 13. The phrase “front three-quarters portion” with regard to the lower denture means about the front three-quarters portion of the bottom surface, illustrated by area L12 in FIG. 14.

The term “periphery” as used herein means the area not greater than about 25 mm from the edge of an upper denture, and not greater than about 10 mm from the edge of a lower denture.

The phrase “peripheral bead” as used herein means a bead of denture adhesive composition within the periphery of an upper or lower denture for a specified distance or area.

The term “safe and effective adhesive amounts” as used herein means an amount sufficient to provide adherence to the gums and/or oral mucous tissues and/or provide adherence of a denture to the gums and/or oral mucous tissues, without toxicity to the denture wearer or damage to the gums and/or oral mucous tissues.

Also, “safe and effective amount”, as used herein, is meant an amount of an agent high enough to significantly (positively) modify the condition to be treated, but low enough to avoid serious side effects (at a reasonable benefit/risk ratio), within the scope of sound medical/dental judgment. The safe and effective amount of an agent may vary with the particular condition being treated, the age and physical condition of the patient being treated, the severity of the condition, the duration of treatment, the nature of concurrent therapy, the specific form of the source employed, and the particular vehicle from which the agent is applied

The percentages used herein to describe the cationic salt function of the alkyl vinyl ether-maleic acid or anhydride copolymers are defined as the stoichiometric percent of the total initial carboxyl groups reacted on the polymer. All percentages and ratios used hereinafter are by weight of total composition, unless otherwise indicated. All measurements referred to herein are made at 25° C. unless otherwise specified. All percentages, ratios, and levels of ingredients referred to herein are based on the actual amount of the ingredient, and do not include solvents, fillers, or other materials with which the ingredient may be combined as a commercially available product, unless otherwise indicated.

A denture adhesive composition may include a denture adhesive component, a non-aqueous denture adhesive carrier, a plasticizer, flavors, fragrances, and sensates. The present invention(s) comprises a safe and effective adhesive amount of a denture adhesive component, generally at a level of from about 10% to about 90%, in another embodiment from about 15% to about 70%, in another embodiment from about 20% to about 50%, and in another embodiment from about 25% to about 45%, by weight of the denture adhesive composition. In one embodiment the denture adhesive composition may comprise at least 20 percent by weight, and in another embodiment at least 30 percent by weight of the denture adhesive composition, of a denture adhesive component.

Denture adhesive components can be any bioadhesive materials and may include natural gums, synthetic polymeric gums, AVE/MA, salts of AVE/MA, AVE/MA IB, salts of AVE/MA/IB, synthetic polymers, mucoadhesive polymers, water-soluble hydrophilic colloids or polymers having the property of swelling upon exposure to moisture to form a mucilaginous mass, hydrophilic polymers, saccharide derivatives, cellulose derivatives, any adhesive material employed in denture stabilizing compositions, and mixtures thereof. Examples of such materials include karaya gum, guar gum, gelatin, algin, sodium alginate, tragacanth, chitosan, polyethylene glycol, polyethylene oxide, acrylamide polymers, polyacrylic acid, homopolymer of acrylic acid cross linked with an allyl ether of pentaerythritol or an allyl ether of sucrose, polyvinyl alcohol, polyamines, polyquarternary compounds, ethylene oxide polymers, polyvinylpyrrolidone, cationic polyacrylamide polymers, AVE/MA, AVE/MA/IB, mixed salts of AVE/MA, mixed salts of AVE/MA/IB, and mixtures thereof.

In one embodiment the denture adhesive component may be AVE/MA, salts of AVE/MA, salts of AVE/MA/IB, mixed salts of AVE/MA, mixed salts of AVE/MA/IB, cellulose derivatives (such as methylcellulose, carboxymethylcellulose, hydroxyethylcellulose, hydroxypropylcellulose, hydroxy-propylmethylcellulose, and mixtures thereof), polyethylene glycol, polyethylene oxide, karaya gum, sodium alginate, chitosan, polyvinyl alcohol, and mixtures thereof. In yet another embodiment, the adhesive component may be AVE/MA, salts of AVE/MA, mixed salts of AVE/MA, cellulose derivatives and mixtures thereof.

In another embodiment the denture adhesive component may be AVE/MA, salts of AVE/MA, salts of AVE/MA/IB, mixed salts of AVE/MA, mixed salts of AVE/MA/IB, karaya gum, sodium alginate, chitosan, polyvinyl alcohol, and mixtures thereof.

In another embodiment the denture adhesive component may be AVE/MA or salts of AVE/MA. The alkyl vinyl ether-maleic acid co-polymer comprises, or consists essentially of, the repeated structural unit: 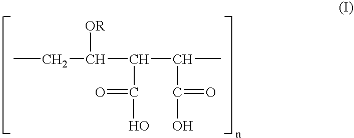
wherein R represents an alkyl radical, including a C₁ to C₅ alkyl radical, n is an integer greater than one representing the number of repeated occurrences of the structural unit in a molecule of the polymer.

In one embodiment, the denture adhesive component may be AVE/MA and salts thereof, including mixed salts of AVE/MA, wherein the copolymer contains a cationic salt function comprising a cation selected from the group consisting of Group IA and Group 2A cations of the periodic table, yttrium, titanium, zirconium, vanadium, chromium, manganese, iron, nickel, copper, zinc, boron, aluminum, cations and mixtures thereof. In another embodiment, the denture adhesive component may be a mixed salt of AVE/MA containing a cationic salt function comprising a cation selected from the group consisting of strontium, zinc, iron, boron, aluminum, vanadium, chromium, manganese, nickel, copper, yttrium, titanium, magnesium, calcium, sodium, cations and mixtures thereof, and in yet another embodiment the cation may be selected from the group consisting of strontium, zinc, iron, magnesium, calcium, sodium, cations, and mixtures thereof.

AVE/MA, in one embodiment, may include a cationic salt function comprising from about 5% to about 50%, in another embodiment, from about 10% to about 40%, in yet another embodiment, from about 10% to about 35% (of the total initial carboxyl groups reacted) zinc cations. These zinc cations can be mixed with other cations selected from the group consisting of strontium, calcium, magnesium and mixtures thereof at a level of from about 5% to about 65%, from about 10% to about 50%. These salts optionally can further comprise from about 0.001% to about 2.5%, from about 0.01% to about 2% of iron, boron, aluminum, vanadium, chromium, manganese, nickel, copper, yttrium, and/or titanium cations.

AVE/MA and salts thereof and AVE/MA/IB and salts thereof, are also described in U.S. Pat. Nos. 5,073,604 to Holeva et al., issued Dec. 17, 1991; U.S. Pat. No. 5,525,652, issued Jun. 11, 1996, Clarke et al.; U.S. Pat. No. 4,758,630, issued Jul. 19, 1988, Shah et al.; U.S. Pat. No. 5,304, 616, issued Apr. 19, 1994, Rajaiah et al.; U.S. Pat. No. 5,424,058, issued Jun. 13, 1995, Rajaiah; U.S. Pat. No. 5,424,058, issued Jun. 13, 1995, Rajaiah et al.; U.S. Pat. No. 4,758,630, issued Jul. 19, 1988, Shah et al.; U.S. Pat. No. 5,830,933, issued Nov. 3, 1998, Synodis et al.; U.S. Pat. No. 2,047,398, issued Jul. 14, 1936, Voss et al.; U.S. Pat. No. 3,003,988, issued Oct. 10, 1961, Germann et al.; U.S. Pat. No. 5,880,172, Rajaiah et al., issued Mar. 9, 1999; U.S. Pat. No. 5,900,470, Prosise et al., issued May 4, 1999; U.S. Pat. No. 5,037,924, Tazi et al., issued Aug. 6. 1991; U.S. Pat. No. 5,082,913, Tazi et al, issued Jan. 21, 1992; U.S. Pat. No. 6,239,191, Wong et al., issued May 29, 2001; U.S. Pat. No. 6,276,937, Gasman et al., issued Aug. 21, 2001; U.S. Pat. No. 6,025,411, Wong et al., issued Feb. 15, 2002. Salts of AVE/MA are also described in P&G patents and copending applications Ser. No. 06/152,158, filed Sep. 2, 1999, Rajaiah et al.; U.S. Pat. No. 6,355,706, 1999, Rajaiah et al.; 60/129,162, filed Apr. 14, 1999, Rajaiah et al.; 60/152,122, filed Sep. 2, 1999, Rajaiah et al.; Ser. No. 09/291,554, filed Apr. 14, 1999, Rajaiah et al.; Ser. No. 09/389,209, filed Sep. 2, 1999, Rajaiah et al.

In one embodiment the free acid level of the salts of the AVE/MA or AVE/MA/IB may be at least about 36%, in another embodiment may be from about 36% to about 60%, and even in another embodiment may be from about 40% to about 55%, of the total initial carboxyl groups of the copolymer or terpolymer.

The specific viscosity of the starting copolymer acid or copolymer anhydride may be from about 1.2 to about 14, when measured in a 1% weight/volume solution in MEK (methyl ethyl ketone) at 25° C. Other methods and solvents can be used to measure the specific viscosity such as a 1% weight/volume solution in DMF (dimethyl formamide) at 25° C. and a 1% weight/volume solution in 2-butanone at 25° C.

Suitable AVE/MA copolymers may be prepared by well-known methods of the prior art; see, for example, U.S. Pat. Nos. 2,782,182 and 2,047,398. Methods of making these mixed salts of AVE/MA polymers are further disclosed in U.S. Pat. Nos. 5,073,604, Holeva et al., issued Dec. 17, 1991 and U.S. Pat. No. 5,872,161, Liang et al., issued Feb. 16, 1999.

The non-aqueous denture adhesive carrier may be a non-aqueous vehicle. The level of non-aqueous vehicle may be from 10% to about 90%, in another embodiment may be from about 20% to about 80%, and in yet another embodiment may be from about 20% to about 60%, by weight of the denture adhesive composition.

The non-aqueous vehicle may generally be any chemical in any physical form that does not contain water. The non-aqueous vehicle may be selected from the group consisting of liquid petrolatum, petrolatum, mineral oil, glycerin, natural and synthetic oils, fats, silicone and silicone derivatives (including silica), polyvinyl acetate, natural and synthetic waxes such as animal waxes like beeswax, lanolin and shellac, hydrocarbons, hydrocarbon derivatives, vegetable oil waxes such as carnauba, candelilla and bayberry wax, vegetable oils such as caprylic/capric triglycerides, in another embodiment may be selected from the group consisting of liquid petrolatum, petrolatum, mineral oil, vegetable oils such as corn, soy bean, cottonseed, castor, palm and coconut oils and animal oil such as fish oil and oleic acid, and mixtures thereof; and in yet another embodiment may be mineral oil.

Vegetable oils comprised of saturated medium chain fatty acids such as caprylic acid, capric acid and mixtures thereof, may be used in the present invention(s). These vegetable oils and other non-aqueous vehicles for denture adhesive compositions are further described in U.S. Pat. No. 5,561,177, issued on Oct. 1, 1996, Khaledi et al.

Other suitable ingredients may include miscellaneous colorants, preservatives (such as methyl and propyl parabens), thickeners such as silicon dioxide, and polyethylene glycol, which may be present at levels of from about 1% to about 20%, by weight of the denture adhesive composition.

Additionally, one or more toxicologically acceptable plasticizers may also be included in the denture adhesive composition. The term “toxicologically-acceptable”, as used herein, is used to describe materials that are suitable in their toxicity profile for administration to humans and/or lower animals. Plasticizers that may be used in the denture adhesive composition may include dimethyl phthalate, diethyl phthalate, dioctyl phthalate, glycerin, diethylene glycol, triethylene glycol, Igepal®, Gafac®, sorbitol, tricresyl phosphate, dimethyl sebacate, ethyl glycolate, ethylphthalyl ethyl glycolate, o- and p-toluene ethyl sulfonamide, and mixtures thereof. Plasticizers may be present at a level of from about 1% to about 70%, in another embodiment from about 1% to about 30%, by weight of the denture adhesive composition.

The denture adhesive composition may also include one or more components which provide flavor, fragrance, and/or sensate benefit (warming or cooling agents). Suitable components include natural or artificial sweetening agents, menthol, menthyl lactate, wintergreen oil, peppermint oil, spearmint oil, leaf alcohol, clove bud oil, anethole, methyl salicylate, eucalyptol, cassia, 1-menthyl acetate, sage, eugenol, parsley oil, oxanone, alpha-irisone, marjoram, lemon, orange, propenyl guaethol, cinnamon, vanillin, thymol, linalool, cinnamaldehyde glycerol acetal known as CGA, and mixtures thereof, as well as coolants. The coolant can be any of a wide variety of materials. Included among such materials are carboxamides, menthol, ketals, diols, and mixtures thereof. Coolants of denture adhesive composition may include the paramenthan carboxyamide agents such as N-ethyl-p-menthan-3-carboxamide, known commercially as “WS-3”, N,2,3-trimethyl-2-isopropylbutanamide, known as “WS-23,” and mixtures thereof. Additional coolants may be selected from the group consisting of menthol, 3-1-menthoxypropane-1,2-diol known as TK-10 manufactured by Takasago, menthone glycerol acetal known as MGA manufactured by Haarmann and Reimer, and menthyl lactate known as Frescolat® manufactured by Haarmann and Reimer. The terms menthol and menthyl as used herein include dextro- and levorotatory isomers of these compounds and racemic mixtures thereof. TK-10 is described in U.S. Pat. No. 4,459,425, Amano et al., issued Jul. 10, 1984. WS-3 and other agents are described in U.S. Pat. No. 4,136,163, Watson, et al., issued Jan. 23, 1979. These agents may be present at a level of from about 1% to about 50%, by weight of the denture adhesive composition.

The denture adhesive composition may also comprise one or more therapeutic actives suitable for topical administration. Therapeutic actives may be present at a level of from about 1% to about 70%, by weight of the denture adhesive composition, and in one embodiment from about 1% to about 20% by weight of the composition. Therapeutic actives may include antimicrobial agents such as iodine, tricolsan, peroxides, sulfonamides, bisbiguanides, or phenolics; antibiotics such as tetracycline, neomycin, kanamycin, metronidazole, or clindamycin; anti-inflammatory agents such as aspirin, acetaminophen, naproxen and its salts, ibuprofen, ketorolac, flurbiprofen, indomethacin, eugenol, or hydrocortisone; dentinal desensitizing agents such as potassium nitrate, strontium chloride or sodium fluoride; fluorides such as sodium fluoride, stannour fluoride, MFP; anesthetic agents such as lidocaine or benzocaine; anti-fungals such as those for the treatment of candida albicans; aromatics such as camphor, eucalyptus oil, and aldehyde derivatives such as benzaldehyde; insulin; steroids; herbal and other plant derived remedies; baking soda, and anti-neoplastics. It is recognized that in certain forms of therapy, combinations of these agents in the same delivery system may be useful in order to obtain an optimal effect. Thus, for example, an antimicrobial and an anti-inflammatory agent may be combined in a single delivery system to provide combined effectiveness.

The viscosity of the denture adhesive composition may be from about 1 Brookfield units to about 100 Brookfield units, from about 5 Brookfield units to about 50 Brookfield units, and from about 10 Brookfield units to about 30 Brookfield units. Herein, Brookfield units are measured at about 25° C. using a Brookfield Viscometer (Model RVT), spindle T-F, at a rotation of 10 r.p.m.

A process for preparing the denture adhesive composition (creams, powders, wafers, non-aqueous liquids, aerosols, pastes) may comprise conventional methods disclosed in the art. Conventional methods are taught in U.S. Pat. No. 5,525,652, issued Jun. 11, 1996, Clarke et al.; U.S. Pat. No. 3,003,988, issued Oct. 10, 1961, Germann et al.; U.S. Pat. No. 5,073,604, Holeva et al., issued Dec. 17, 1991; and U.S. Pat. No. 5,872,161, Liang et al., issued Feb. 16, 1999.

Generally, cream, paste, aerosols, and non-aqueous liquid denture adhesive compositions may be applied to the top surface of the upper denture and/or the bottom surface of the lower denture and thereafter appropriately secured to the gums and/or mucous tissues.

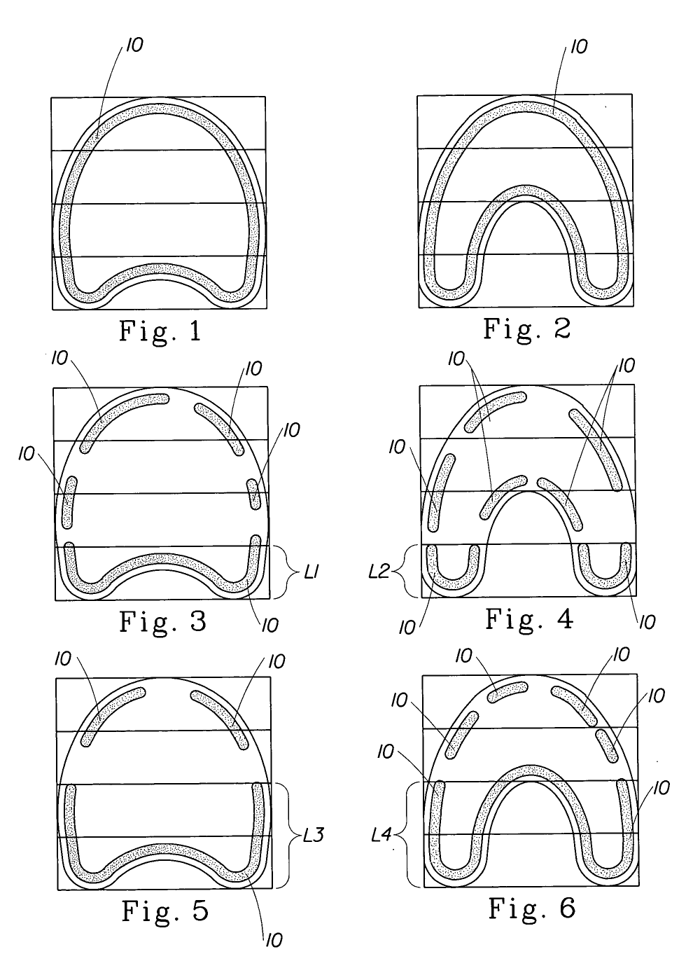

As shown in FIGS. 1 and 2, the denture adhesive composition may be applied as a continuous peripheral bead 10 around the entire upper and lower dentures such that an unbroken ring is formed around the upper denture and the lower denture. That is, the denture adhesive composition may be applied as a continuous bead around the entire periphery of the upper and lower dentures.

As shown in FIGS. 3 and 4, the denture adhesive composition may be applied as a continuous peripheral bead 10 around the back quarter portion, area L1, of the upper denture and the back quarter portions, area L2, of the lower denture.

As shown in FIGS. 5 and 6, the denture adhesive composition may be applied as a continuous peripheral bead 10 around the back half portion, area L3, of the upper denture and the back half portion, area L4, of the lower denture.

As shown in FIGS. 7 and 8, the denture adhesive composition may be applied as a continuous peripheral bead 10 around the back three-quarters portion, area L5, of the upper denture and the back three-quarters portion, area L6, of the lower denture.

As shown in FIGS. 9 and 10, the denture adhesive composition may be applied as a continuous peripheral bead 10 around the front quarter portion, area L7, of the upper denture and the front quarter portion, area L8, of the lower denture.

As shown in FIGS. 11 and 12, the denture adhesive composition may be applied as a continuous peripheral bead 10 around the front half portion, area L9, of the upper denture and the front half portion, area L10, of the lower denture.

As shown in FIGS. 13 and 14, the denture adhesive composition may be applied as a continuous peripheral bead 10 around the front three-quarters portion, area L11, of the upper denture and the front three-quarters portion, area L12, of the lower denture.

The peripheral bead 10 applied to the upper denture may be from about 0.1 g to about 4 g, from about 0.2 g to about 1 g, from about 0.3 g to about 0.8 g; and the peripheral bead 10 applied to the lower denture may be from about 0.1 g to about 4 g, from about 0.1 g to about 1.5 g, from about 0.2 g to about 0.6 g. The peripheral bead 10 may have a thickness from about 0.1 mm to about 3.8 mm, from about 0.5 mm to about 3.2 mm, from about 0.6 mm to about 2.5 mm, from about 0.8 mm to about 1.5 mm, from about 1 mm to about 2 mm.

As shown in FIGS. 15 and 16, an additional non-peripheral bead 12 (that is, a bead not within the periphery of a denture) of denture adhesive composition may be applied to the top surface of the upper denture and/or the bottom surface of the lower denture in a manner that is necessary to achieve desired hold.

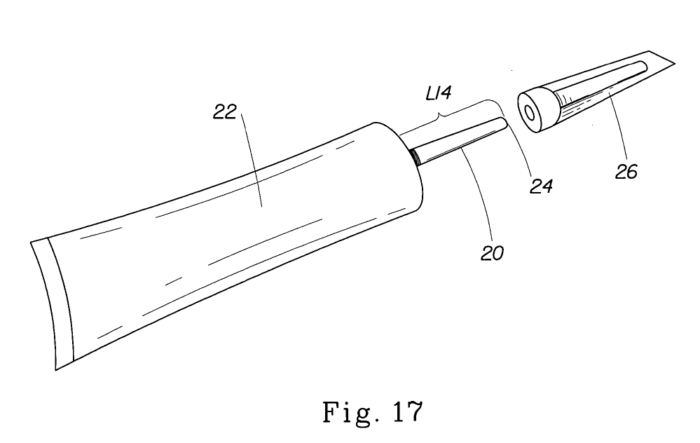

As shown in FIG. 17, a nozzle 20 may be integrally formed with or fitted to a container 22 (e.g., a plastic or metal tube) containing denture adhesive composition. The nozzle 20 may be used for dispensing the above-mentioned denture adhesive compositions. The nozzle 20 may have an open end 24 for dispensing the denture adhesive composition. The nozzle 20 may have a length, distance L14, from about 5 mm to about 25 mm, from about 5 mm to about 20 mm, from about 5 mm to about 15 mm. The open end 24 may have a diameter from about 0.1 mm to about 4 mm, from about 1 mm to about 3 mm. The nozzle 20 may be threaded for fastening a lid 26 to. Alternatively, the lid 26 may be friction fitted, snapped, etc. Also, a flip cap (not shown) may be used to seal the open end 24.

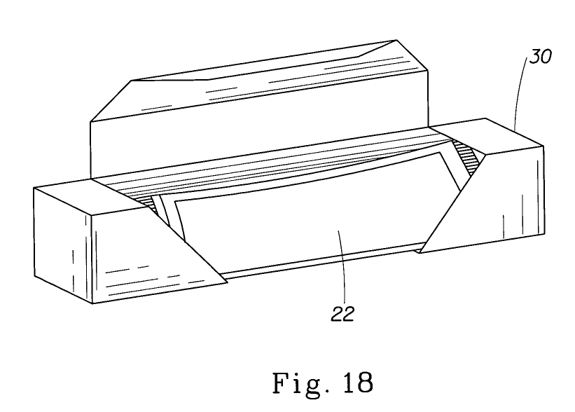

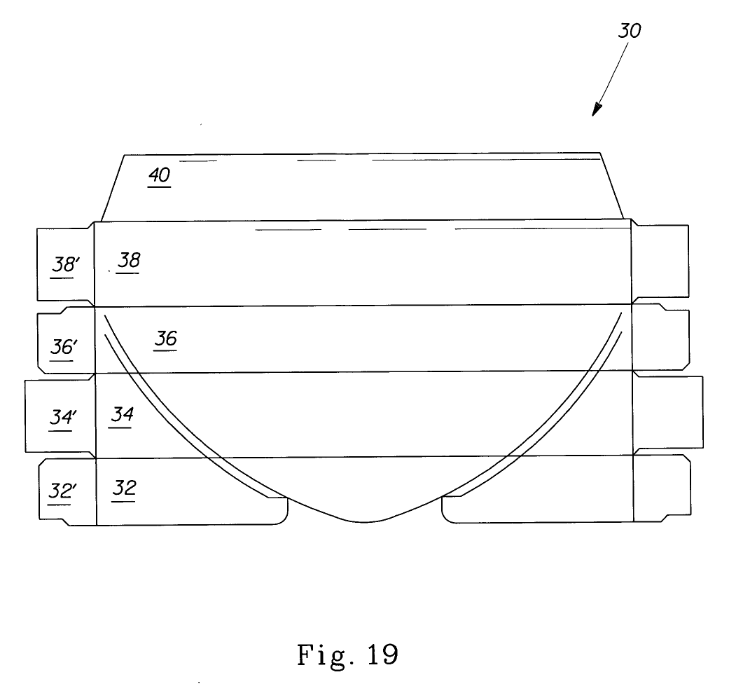

As shown in FIG. 18, a box 30 may be used for holding the container 22 of denture adhesive composition having a nozzle 20. As shown in FIG. 19, the box 30 may comprise a single piece of material (e.g., cardboard, plastic, etc.) which comprises an outer bottom panel 32, a front panel 34, a top panel 36, a back panel 38, and an inner bottom panel 40. These panels may be rectangular, and defined by seams in a single piece of box 30 material.

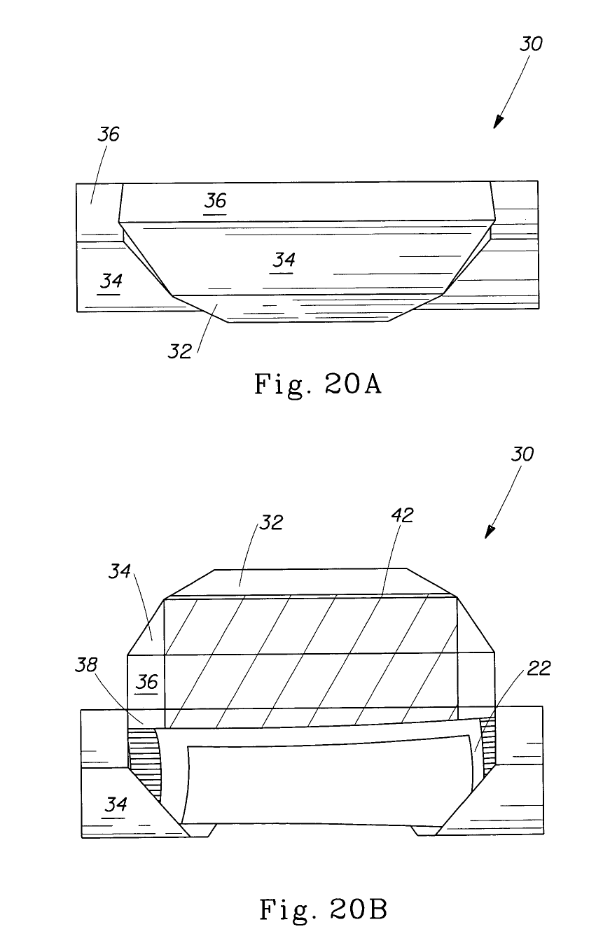

An enclosed volume may be formed by folding each of the panels at a 90 angle, overlapping tabs (32′, 34′, 36′, and 38′) on one another. As shown in FIGS. 20-A and 20-B, the outer bottom panel 32, the front panel 34, and the top panel 36 may be perforated such that the box 30 may be opened in a manner revealing a large portion of the box 30 interior, while still holding the container 22 of denture adhesive composition and nozzle 20 in place.

The exposed interior space (that is, the inside of the outer bottom panel 32, the front panel 34, the top panel 36, and the back panel 38) of the box 30 may be used to provide information 42 (instructions (text and or diagrams), advertisements, etc.). Actually, most all of the interior space of the box 30 may be used. The information 42 may be printed and/or fixed onto the box 30. Additionally, information 42 may also be printed and/or fixed to any exterior portion of the box 30.

The interior surface area for information 42 may be about the same as the exterior surface area for information 42. Thus, information 42 may be written in a larger font (to assist people with compromised vision), and diagrams can be more easily used as the inside space of the outer bottom panel 32, the front panel 34, and the top panel 36 can be unfolded such that they are, in addition to the back panel 38, essentially in the same vertical plane (see FIG. 20-B).

The following examples further describe and demonstrate embodiments within the scope of the present invention(s). The examples are given solely for the purpose of illustration and are not to be construed as limitations of the present invention(s). Many variations of these are possible without departing from the spirit and scope of the invention.

### EXAMPLE I¹

A denture wearer applies about 0.1 g to about 4 g of any of the denture adhesive compositions described below as a peripheral bead 10 to the top surface of the upper denture in the configuration illustrated in FIG. 1, applies about 0.1 g to about 4 g of any of the denture-adhesive compositions described below as a peripheral bead 10 to the bottom surface of the lower denture in the configuration illustrated in FIG. 2. Then the subject inserts the denture(s) into his/her mouth and presses it into place. After applying the denture adhesive composition, the denture adhesive composition prevents, reduces, and inhibits calculus, tartar, plaque, stain and/or microbes on the gums and/or oral mucous tissues. The denture adhesive composition also prevents, reduces, and inhibits entry of food between the denture and the soft tissue of the wearer.
¹The compositions of Table 1 may be modified by increasing or decreasing the level of AVE/MA salts, carboxymethylcellulose, petrolatum, and mineral oil by 0 to 15 grams.

TABLE 1

| | A | B | C | D | E | F | G | H | I | J | K | L |
|---|---|---|---|---|---|---|---|---|---|---|---|---|
| | Grams | Grams | Grams | Grams | Grams | Grams | Grams | Grams | Grams | Grams | Grams | Grams |
| Mineral Oil | 23.93 | 23.93 | 23.93 | 23.93 | 23.93 | 23.93 | 23.93 | 16 | 16 | 16 | 16 | 16 |
| (e.g., white) | | | | | | | | | | | | |
| Petrolatum | 21.87 | 24.87 | 27.87 | 29.87 | 37.87 | 21.87 | 19.87 | 27.85 | 33.35 | 36.85 | 40.85 | 31.85 |
| (e.g., white) | | | | | | | | | | | | |
| Carboxymethyl- | 20 | 20 | 20 | 20 | 15 | 20 | 20 | 24 | 22 | 20 | 18 | 20 |
| cellulose | | | | | | | | | | | | |
| Silica | 1.14 | 1.14 | 1.14 | 1.14 | 1.14 | 1.14 | 1.14 | 1.1 | 1.1 | 1.1 | 1.1 | 1.1 |
| Colorant (e.g., D&C | 0.06 | 0.06 | 0.06 | 0.06 | 0.06 | 0.06 | 0.06 | 0.05 | 0.05 | 0.05 | 0.05 | 0.05 |
| Red 27 (CAS | | | | | | | | | | | | |
| 13473-26-2)) | | | | | | | | | | | | |
| Calcium/Zinc Salt or | 33 | 30 | 27 | 25 | 22 | 32 | 35 | 30 | 27 | 25 | 22 | 30 |
| Magnesium/Zinc/ | | | | | | | | | | | | |
| Sodium or Calcium/ | | | | | | | | | | | | |
| Sodium or mixture | | | | | | | | | | | | |
| thereof, of AVE/MA | | | | | | | | | | | | |
| or AVE/MA/IB | | | | | | | | | | | | |
| Any Acid of AVE/MA | 0 | 0 | 0 | 0 | 0 | 1 | 0 | 1 | 0.5 | 1 | 2 | 1 |
| and/or AVE/MA/IB | | | | | | | | | | | | |
| | 100 | 100 | 100 | 100 | 100 | 100 | 100 | 100 | 100 | 100 | 100 | 100 |

The colorant and non-aqueous carriers (mineral oil and petrolatum) are weighed, heated and mixed in a glass jar at 50° C. to 60° C. until visually uniform. Then the powders (silica, CMC, AVE/MA) are weighed and shake-blended together in a container. Thereafter, the powders are mixed into the non-aqueous carriers with a spatula until visually a uniform pink cream.

All documents cited herein are incorporated by reference; the citation of any document is not to be construed as an admission that it is prior art with respect to the present invention(s).

While particular embodiments of the present invention(s) have been illustrated and described, it would be obvious to those skilled in the art that various other changes and modifications can be made without departing from the spirit and scope of the invention. It is therefore intended to cover in the appended claims all such changes and modifications that are within the scope of this invention.
² Colorant may be in dye, lake, dispersion, pigment and/or any other form.

1. A denture care kit for decreasing food entrapment comprising:
a. a denture adhesive composition comprising a safe and effective adhesive amount of a denture adhesive component, wherein said denture adhesive composition has a viscosity of from about 5 to about 80 Brookfield;
b. a container for holding and dispensing said denture adhesive composition, said container comprising a nozzle; and
c. information for use for decreasing food entrapment which instructs the denture wearer to apply said denture adhesive composition as a continuous peripheral bead along at least one of a back quarter portion, a back half portion, a back three-quarters portion, a front quarter portion, a front half portion, a front three-quarters portion, or an entire periphery of an upper or lower denture.

2. The denture care kit of claim 1, wherein said nozzle has a tip diameter of from about 80 mils to about 120 mils or has a nozzle tip open surface area of from about 5,024 square mils to about 11,304 square mils, and wherein said nozzle has a length of from about 10 mm to about 25 mm.

3. The denture care kit of claim 1, wherein said nozzle has a tip diameter from about 90 mils to about 110 mils or has a nozzle tip open surface area of from about 6,359 square mils to about 9,499 square mil, and wherein said nozzle has a length of from about 10 mm to about 25 mm.

4. The denture care kit of claim 3, wherein said denture adhesive composition has a viscosity of from about 10 to about 50 Brookfield.

5. The denture care kit of claim 4, wherein said denture adhesive component is selected from the group consisting of AVE/MA, mixed salts of AVE/MA, AVE/MA/IB, mixed salts of AVE/MA/IB, cellulose derivatives, and mixtures thereof.

6. The denture care kit of claim 5, wherein said denture adhesive component is selected from the group consisting of AVE/MA, mixed salts of AVE/MA, cellulose derivatives, and mixtures thereof.

7. The denture care kit of claim 1, wherein said information for use further instructs the denture wearer to apply said denture adhesive composition within a non-peripheral area of said denture.

8. The denture care kit of claim 7, wherein said information for use further instructs the denture wearer to apply said denture adhesive composition to a central portion of said upper or lower denture.

9. The denture care kit of claim 1, wherein the denture care kit further comprises a box for holding said container, said box having said information fixed or printed at least within an interior portion of said box, wherein said information may be viewed upon opening said box.

10. A method of decreasing food entrapment comprising:
a. opening a box having information fixed or printed within an interior portion of said box, said information for instructing a denture wearer how to apply a denture adhesive composition along a periphery of a denture in a manner that will decrease food entrapment between said denture and the denture wearer's gums or oral mucous tissues;
b. removing a tube from said box, said tube containing said denture adhesive composition, said denture adhesive composition having a viscosity of from about 5 to about 80 Brookfield; and
c. applying a continuous peripheral bead of said denture adhesive composition to an upper or a lower denture in substantial conformity with said information.

11. The method of claim 10, wherein said information for use instructs the denture wearer to apply said denture adhesive composition as a continuous peripheral bead along at least one of a back quarter portion, a back half portion, a back three-quarters portion, a front quarter portion, a front half portion, a front three-quarters portion, or an entire periphery of an upper or lower denture.

12. The method of claim 10, wherein said information instructs the denture wearer to apply said denture adhesive compositions around the entire upper or lower denture.

13. The method of claim 11, wherein said denture wearer applies a continuous bead from about 1 mm to about 15 mm from the edge of said upper denture.

14. The method of claim 11, wherein said denture wearer applies a continuous bead from about 1 mm to about 5 mm from the edge of said lower denture.

15. The method of claim 14, wherein said nozzle has a tip diameter of from about 80 mils to about 120 mils or has a nozzle tip open surface area of from about 5,024 square mils to about 11,304 square mils, and wherein said nozzle has a length of from about 10 mm to about 25 mm.

16. The method of claim 15, wherein said denture adhesive composition has a viscosity of from about 15 to about 30 Brookfield.

17. The method of claim 10, wherein said denture adhesive composition comprises mixed salts of AVE/MA, cellulose derivatives, petrolatum, mineral oil, and silica.

18. The method of claim 17, wherein said denture adhesive composition further comprises AVE/MA acid.

19. The method of claim 11, wherein said information is fixed or printed on both said interior and exterior portions of said box, wherein said information on said interior portion of said box is a larger font than said information on said exterior of said box.

20. A denture care kit for improving hold comprising:
a. a denture adhesive composition comprising a safe and effective adhesive amount of a denture adhesive component, wherein said denture adhesive composition has a viscosity of from about 5 to about 80 Brookfield;
b. a container for holding and dispensing said denture adhesive composition, said container comprising a nozzle; and
c. information for use which instructs a denture wearer to apply said denture adhesive composition as a peripheral bead along at least one of a back quarter portion, a front half portion, a front three-quarters portion, or an entire periphery of an upper or lower denture, wherein said information also instructs the denture wearer to apply said denture adhesive composition within a non-peripheral area of an upper or lower denture.

21. The denture care kit of claim 20, wherein said peripheral bead is continuous.

22. The denture care kit of claim 21, wherein said information for use instructs the denture wearer to apply said denture adhesive composition around said entire periphery of said upper denture.

23. The denture care kit of claim 21, wherein said nozzle has a tip diameter of from about 80 mils to about 120 mils or has a nozzle tip open surface area of from about 5,024 square mils to about 11,304 square mils, and wherein said nozzle has length has a length of from about 10 mm to about 25 mm.

24. The denture care kit of claim 22, wherein said information for use further instructs the denture wearer to apply said denture adhesive composition for decreasing food entrapment.

25. The denture care kit of claim 22, wherein said information for use instructs the denture wearer to apply said denture adhesive composition around said entire periphery of said upper denture.

26. The denture care kit of claim 25, wherein said information for use instructs the denture wearer to apply the denture adhesive composition as two generally parallel non-peripheral beads in said non-peripheral area of said upper denture, wherein said non-peripheral beads are oriented generally transverse to a longitudinal axis of said upper denture.

27. The denture care kit of claim 25, wherein said information for use instructs the denture wearer to apply said denture composition as a continuous non-peripheral bead in said non-peripheral area of said lower denture, wherein said non-peripheral bead is oriented generally evenly between said peripheral bead.

28. The denture care kit of claim 21, wherein said information for use instructs the denture wearer to apply said denture adhesive composition around said back quarter portion of said upper denture.

29. A method of decreasing food entrapment or sealing out food from under an upper or lower denture, the method comprising:
a. applying a safe and effective amount of a denture adhesive composition on at least a back quarter portion of said upper or lower denture as a continuous peripheral bead for decreasing food entrapment or sealing out food from under said upper or lower denture; and
b. inserting said upper or lower denture into the oral cavity of a denture wearer in need thereof.

30. The method of claim 29, wherein method further comprises applying said denture adhesive composition to at least one of a back half portion, a back three-quarters portion, a front quarter portion, a front half portion, a front three-quarters portion, or an entire periphery of an upper or lower denture.

31. The method of claim 29, wherein said method further comprises applying said denture adhesive composition to a non-peripheral area of said upper or lower denture.

32. The method of claim 31, wherein said method further comprises applying said denture adhesive composition as two generally parallel non-peripheral beads to said non-peripheral area of said upper denture for improved hold, wherein said non-peripheral beads are oriented generally transverse to a longitudinal axis of said upper denture.

33. The method of claim 31, wherein said method further comprises applying said denture adhesive composition as a continuous non-peripheral bead to said non-peripheral area of said lower denture for improved hole.

34. The method of claim 29, wherein said denture adhesive composition is applied from about 1 mm to about 15 mm from the edge of said upper or lower denture.

35. The method of claim 30, wherein said denture adhesive composition is applied from about 1 mm to about 5 mm from the edge of said upper or lower denture.

36. The method of claim 30, wherein said method further comprises applying a continuous bead of said denture adhesive composition around said entire periphery of said lower denture, and applying a non-peripheral bead of said denture adhesive composition in a non-peripheral area of said lower denture, wherein said non-peripheral bead is oriented generally evenly between said peripheral bead.
